(19) United States
(12) Patent Application Publication
(10) Pub. No.: US 2025/0134806 A1
(43) Pub. Date: May 1, 2025

(54) PHARMACEUTICAL COMPOSITION FOR DRY POWDER INHALATION AND PREPARATION METHOD THEREOF

(71) Applicant: ASG INSPIRATION LABORATORY (SINGAPORE) PTE. LTD., SINGAPORE (SG)

(72) Inventors: Sung Tsai YU, Taipei City (TW); Hsin-Ke LI, Taipei City (TW); Hao-Wei HUANG, Taipei City (TW); Shih-Tan CHEN, Taipei City (TW); Hou In LEONG, Taipei City (TW); Johnson LEE, Taipei City (TW); Jao Juen HUNG, Taipei City (TW); Shih Pan CHAO, Taipei City (TW); Wen-Hui FU, Taipei City (TW)

(21) Appl. No.: 18/931,074

(22) Filed: Oct. 30, 2024

Related U.S. Application Data
(60) Provisional application No. 63/594,959, filed on Nov. 1, 2023.

(51) Int. Cl. A61K 9/00; A61K 9/16; A61K 31/4985

(52) U.S. Cl. CPC A61K 9/0075; A61K 9/1623; A61K 9/1652; A61K 31/4985

(57) ABSTRACT

A pharmaceutical composition for dry powder inhalation is provided, including an active ingredient and a first pharmacologically acceptable excipient. The active ingredient includes tadalafil or a pharmaceutically acceptable salt thereof. The first pharmacologically acceptable excipient includes amino acid, polysaccharide, phospholipid, polylactic acid, polylactic acid copolymer, or a combination thereof. In some embodiments of the present disclosure, a method of preparing a pharmaceutical composition for dry powder inhalation is further provided. The pharmaceutical composition for dry powder inhalation increases aerosol property meets the requirements for inhalation administration and reduces onset time.

### CROSS-REFERENCE TO RELATED APPLICATION

This application claims priority to U.S. Provisional Patent Application No. 63/594,959, filed Nov. 1, 2023, the disclosure of which is hereby incorporated by reference in its entirety.

### BACKGROUND OF THE INVENTION

### Field of Invention

The present disclosure relates to a pharmaceutical composition for dry powder inhalation and a preparation thereof. In particular, the present disclosure relates to a pharmaceutical composition including tadalafil or a pharmaceutically acceptable salt thereof.

### Description of Related Art

Tadalafil, an inhibitor of phosphodiesterase type 5 (PDE5), is mainly used for treating male penile Erectile Dysfunction (ED), and is one of the mainstream medicaments for treating ED at present. Currently, tadalafil is traditionally administered orally, which limits the onset time since tadalafil needs to be absorbed through the gastrointestinal tract before it reaches the blood circulation.

Therefore, how to provide a pharmaceutical composition containing tadalafil and suitable for dry powder inhalation to increase onset time of tadalafil remains to be solved.

### SUMMARY OF THE INVENTION

In one aspect of the present disclosure, a pharmaceutical composition for dry powder inhalation is provided, including: an active ingredient and a first pharmacologically acceptable excipient. The active ingredient includes tadalafil or a pharmaceutically acceptable salt thereof. The first pharmacologically acceptable excipient includes amino acid, polysaccharide, phospholipid, polylactic acid, polylactic acid copolymer, or a combination thereof.

In some embodiments, a weight percentage of the active ingredient is from 1% to 99% and a weight percentage of the first pharmacologically acceptable excipient is from 1% to 99% based on 100% by weight of the active ingredient and the first pharmacologically acceptable excipient.

In some embodiments, a weight percentage of the active ingredient is from 1% to 99%, and a weight percentage of the amino acid is from 1% to 99% based on 100% by weight of the active ingredient and the first pharmacologically acceptable excipient.

In some embodiments, the amino acid includes glycine, alanine, valine, leucine, isoleucine, phenylalanine, tryptophan, tyrosine, aspartic acid, histidine, asparagine, glutamic acid, lysine, glutamine, methionine, arginine, serine, threonine, cysteine, proline or a combination thereof.

In some embodiments, a weight percentage of the active ingredient is from 1% to 99%, and a weight percentage of the polysaccharide is from 1% to 99% based on 100% by weight of the active ingredient and the first pharmacologically acceptable excipient.

In some embodiments, the polysaccharide includes chitosan, chitosan salt, chitosan glutamate, hyaluronic acid or a combination thereof.

In some embodiments, a weight percentage of the active ingredient is from 1% to 99%, and a weight percentage of the phospholipid is from 1% to 99% based on 100% by weight of the active ingredient and the first pharmacologically acceptable excipient.

In some embodiments, the phospholipid includes dipalmitoyl phosphatidylcholine, distearoyl phosphatidyl choline or a combination thereof.

In some embodiments, a weight percentage of the active ingredient is from 1% to 99%, and a weight percentage of the polylactic acid is from 1% to 50% based on 100% by weight of the active ingredient and the first pharmacologically acceptable excipient.

In some embodiments, a weight percentage of the active ingredient is from 1% to 99%, and a weight percentage of the polylactic acid copolymer is from 1% to 50% based on 100% by weight of the active ingredient and the first pharmacologically acceptable excipient.

In some embodiments, the polylactic acid copolymer includes poly(lactic-co-glycolic acid).

In some embodiments, the active ingredient and the first pharmacologically acceptable excipient forms a finely divided particle having a particle size of from 50 nm to 10 μm.

In some embodiments, the finely divided particle is provided in a solid spherical-shaped form, a hollow spherical-shaped form, a solid polyhedron form or a combination thereof.

In some embodiments, the pharmaceutical composition further includes a second pharmacologically acceptable excipient different from the first pharmacologically acceptable excipient.

In some embodiments, a total weight percentage of the active ingredient and the first pharmacologically acceptable excipient is from 0.005% to 30%, and a weight percentage of the second pharmacologically acceptable excipient is from 70% to 99.995% based on 100% by weight of the pharmaceutical composition.

In some embodiments, the second pharmacologically acceptable excipient includes lactose, mannitol or a combination thereof.

In another aspect of the present disclosure, a method of preparing a pharmaceutical composition for dry powder inhalation, including: dissolving an active ingredient in a first solvent to form a first solution, wherein the active ingredient includes tadalafil or a pharmaceutically acceptable salt thereof; dissolving a first pharmacologically acceptable excipient in a second solvent to form a second solution, wherein the first pharmacologically acceptable excipient includes amino acid, polysaccharide, phospholipid, polylactic acid, polylactic acid copolymer, or a combination thereof; mixing the first solution and the second solution to form a mixture; and spray drying the mixture to form a finely divided particle.

In some embodiments, the first solvent and the second solvent includes a first organic solvent, and the second solvent comprises a second organic solvent, water or a combination thereof.

In some embodiments, a weight percentage of the active ingredient and the first pharmacologically acceptable excipient is from 0.5% to 3% based on 100% by weight of the mixture.

In some embodiments, a weight ratio of the active ingredient and the first pharmacologically acceptable excipient in the mixture is from 0.01:1 to 199:1.

In some embodiments, spray drying the mixture is performed at an outlet temperature of from 35° C. to 110° C.

In some embodiments, the method further includes mixing the finely divided particle with a second pharmacologically acceptable excipient different from the first pharmacologically acceptable excipient.

In some embodiments, the second pharmacologically acceptable excipient includes a first size group, a second size group or a combination thereof, wherein a volume-basis particle size distribution of the first size group is different from a volume-basis particle size distribution of the second size group.

In some embodiments, a particle size D50 of the first size group is from 5 μm to 50 μm and a particle size D50 of the second size group is from 30 μm to 125 μm.

In some embodiments, the method further includes mixing the finely divided particle with a flavoring agent.

### BRIEF DESCRIPTION OF THE DRAWINGS

In order to make the above-mentioned and other objects, features, advantages and embodiments of the present disclosure more clearly understood, descriptions of accompanying drawings are as follows:

FIG. 1 illustrates a flow chart of a method of preparing a pharmaceutical composition for dry powder inhalation in some embodiments of the present disclosure.

FIG. 2 illustrates tadalafil without spray drying under the field of an electron microscope.

FIG. 3 illustrates finely divided particles under the field of an electron microscope, in which tadalafil is mixed with leucine before spray drying.

FIG. 4A illustrates finely divided particles under the field of an electron microscope, in which tadalafil is mixed with chitosan glutamate before spray drying.

FIG. 4B illustrates finely divided particles under the field of an electron microscope, in which tadalafil is mixed with hyaluronic acid before spray drying.

FIG. 5 illustrates finely divided particles under the field of an electron microscope, in which tadalafil is mixed with distearoyl phosphatidyl choline (DSPC) before spray drying.

FIG. 6 illustrates aerosol properties of group Tad only, group Tad+Leucine and group Tad+Leucine+Lactose detected by using Next Generation Impactor (NGI).

### DESCRIPTION OF THE INVENTION

In order that the present disclosure is described in detail and completeness, implementation aspects and specific embodiments of the present disclosure with illustrative description are presented, but those are not the only form for implementation or plucuse of the specific embodiments of the present disclosure. The embodiments disclosed herein may be combined or substituted with each other in an advantageous manner, and other embodiments may be added to an embodiment without further description. In the following description, numerous specific details will be described in detail in order to enable the reader to fully understand the following embodiments. However, the embodiments of the present disclosure may be practiced without these specific details.

Although a series of operations or steps are described below to illustrate the method disclosed herein, the order of the operations or steps is not to be construed as limiting. For example, certain operations or steps may be performed in a different order and/or concurrently with other steps. In addition, not all illustrated operations, steps, and/or features are required to implement embodiments of the present disclosure. Moreover, each of the operations or steps described herein may include a plurality of sub-steps or actions.

In this description, unless the context specifically dictates otherwise, “a” and “the” may mean a single or a plurality. It will be further understood that “comprise”, “include”, “have”, and similar terms as used herein indicate described features, regions, integers, steps, operations, elements and/or components, but not exclude other features, regions, integers, steps, operations, elements, components and/or groups.

As used herein, “drug” or “active ingredient” refers to tadalafil or its pharmaceutically acceptable salt thereof, including but not limited to salts, esters, complexes, chelating agents, cage compounds, racemates, mirror image isomers, or the like.

As used herein, “pharmacologically acceptable excipient” refers to pharmaceutical additives without pharmacological activity and used in pharmaceutical compositions according to different purposes and functions.

The main purpose of the present disclosure is to provide a pharmaceutical composition for dry powder inhalation with reduced particle sizes and some specified shapes, thereby increasing aerosol property (aerodynamics, such as fine particle fraction (FPF)) for meeting the requirements for inhalation administration and reducing onset time.

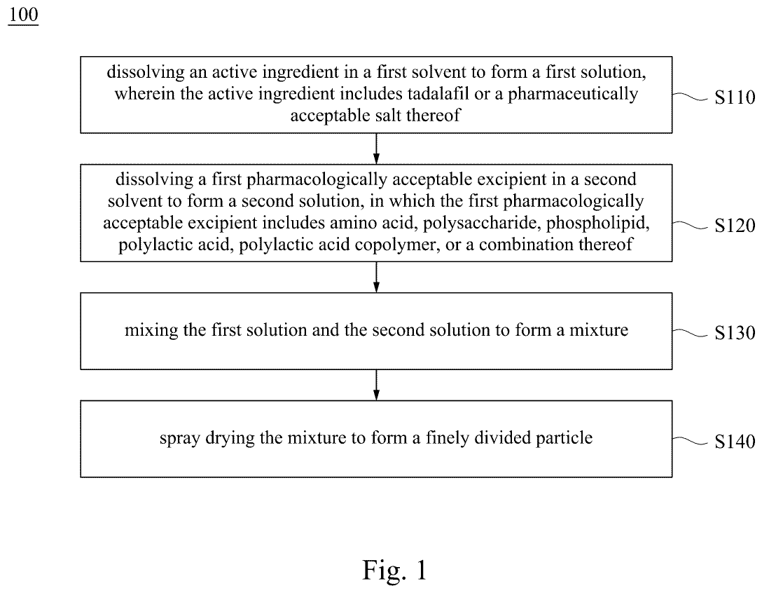

Please refer to FIG. 1, representing a flow chart of a method 100 of preparing a pharmaceutical composition for dry powder inhalation in some embodiments of the present disclosure, including step S110, step S120, step S130 and step S140. It should be emphasized that by mixing a first pharmacologically acceptable excipient with tadalafil in the mixture and then spray drying the mixture, the shape and the particle size of finely divided particles can be regulated (including but not limited that the shape appears to be spherical-shaped form or polyhedron form, and the particle sizes are more consistent in different lots), so that the aerosol property (such as longer flight distance) and onset time of the finely divided particle can be improved.

First of all, refer to step S110, dissolving an active ingredient in a first solvent to form a first solution, in which the active ingredient includes tadalafil or a pharmaceutically acceptable salt thereof.

In some embodiments, the first solution includes a first organic solvent for dissolving the active ingredient much easily, such as ethanol, methanol, methylene chloride, ethyl acetate, acetonitrile, acetone, dimethyl sulfoxide or a combination thereof. In some other embodiments, the first solution includes water.

Please refer to step S120, dissolving a first pharmacologically acceptable excipient in a second solvent to form a second solution, in which the first pharmacologically acceptable excipient includes amino acid, polysaccharide, phospholipid, polylactic acid, polylactic acid copolymer, or a combination thereof.

In some embodiments, the second solution includes a second organic solvent or water for dissolving the first pharmacologically acceptable excipient much easily. In some embodiments, the second organic solvent includes ethanol, methanol, methylene chloride, ethyl acetate, acetonitrile, acetone, dimethyl sulfoxide or a combination thereof. In some embodiments, the second solution is the same as the first solution.

In some embodiments, amino acid includes glycine, alanine, valine, leucine, isoleucine, phenylalanine, tryptophan, tyrosine, aspartic acid, histidine, asparagine, glutamic acid, lysine, glutamine, methionine, arginine, serine, threonine, cysteine, proline or a combination thereof. In some embodiments, polysaccharide includes chitosan, chitosan salt, chitosan glutamate, hyaluronic acid or a combination thereof. In some embodiments, phospholipid includes dipalmitoyl phosphatidylcholine (DPPC), distearoyl phosphatidyl choline (DSPC) or a combination thereof. In some embodiments, polylactic acid copolymer includes poly(lactic-co-glycolic acid) (PLGA).

Please refer to step S130, mixing the first solution and the second solution to form a mixture.

In some embodiments, a weight percentage of the active ingredient and the first pharmacologically acceptable excipient is from 0.5% to 3% by weight of the mixture, such as, 0.5%, 1%, 1.5%, 2%, 2.5%, 3% or any value between any interval of the abovementioned values. If the weight percentage is too low, the production yield for spray drying is limited. If the weight percentage is too high, the efficiency of spray drying is limited since the mixture may be uneven and too viscose to be spray dried. In some embodiments, a weight ratio of the active ingredient and the first pharmacologically acceptable excipient in the mixture is from 0.01:1 to 199:1, such as 0.01:1, 0.1:1, 1:1, 10:1, 20:1, 30:1, 40:1, 50:1, 60:1, 70:1, 80:1, 90:1, 100:1, 110:1, 120:1, 130:1, 140:1, 150:1, 160:1, 170:1, 180:1, 190:1, 199:1 or any value between any interval of the abovementioned values. If the weight ratio is too low, the yield of the active ingredient contained in the finely divided particle after spray drying is limited. If the weight ratio is too high, the aerosol property is reduced since the finely divided particle is hardly regulated by the first pharmacologically acceptable excipient.

Please refer to step S140, spray drying the mixture to form a finely divided particle.

In some embodiments, spray drying the mixture is performed at an outlet temperature of from 35° C. to 110° C., such as 35° C., 40° C., 45° C., 50° C., 55° C., 60° C., 65° C., 70° C., 75° C., 80° C., 85° C., 90° C., 95° C., 100° C., 110° C. or any value between any interval of the abovementioned values. If the outlet temperature is too low, the sprayed droplets are too large, the particle size of the sprayed particles tends to be larger, and the shape of the sprayed particles will be difficult to maintain in a spherical-shaped form or a polyhedron form, tending to be irregular. If the outlet temperature is high, the structure of the active ingredient or the pharmacologically acceptable excipients may be changed, thereby influencing the functions.

In some embodiments, the finely divided particle can be packed into capsules, aluminum foil blisters, and drug storage tanks in dry powder inhalation devices for subjects in need to inhale.

In some embodiments, the method 100 further includes mixing the finely divided particle with a second pharmacologically acceptable excipient different from the first pharmacologically acceptable excipient. It's noted that the addition of the second pharmacologically acceptable excipient increases the aerosol property, prolonging the flight distance of the finely divided particle and increasing the distribution ratio in lungs after inhalation administration.

In some embodiments, the second pharmacologically acceptable excipient includes lactose, mannitol or a combination thereof.

In some embodiments, the second pharmacologically acceptable excipient includes a first size group, a second size group or a combination thereof, in which a volume-basis particle size distribution of the first size group is different from a volume-basis particle size distribution of the second size group. In some embodiments, a particle size D50 of the first size group is from 5 μm to 50 μm (5 μm, 10 μm, 20 μm, 30 μm, 40 μm, 50 μm or any value between any interval of the abovementioned values) and a particle size D50 of the second size group is from 30 μm to 125 μm (30 μm, 40 μm, 50 μm, 60 μm, 70 μm, 80 μm, 90 μm, 100 μm, 110 μm, 125 μm or any value between any interval of the abovementioned values).

In some embodiments, a weight ratio of the second pharmacologically acceptable excipient and the finely divided particle is from 70:30 to 99.995:0.005, such as 70:30, 80:20, 90:10, 99.995:0.005 or any value between any interval of the abovementioned values. If the weight ratio of the second pharmacologically acceptable excipient is too high, the active ingredient provided in a specific unit of pharmaceutical composition is limited. If the weight ratio of the second pharmacologically acceptable excipient is too low, the improvement of the aerosol property is limited.

In some embodiments, the method 100 further includes mixing the finely divided particle with a flavoring agent (such as menthol or natural flavors tasting like lemon, strawberry, orange, etc.) of a weight percentage of less than 1% (such as 0.1%, 0.5%, 1% or any value between any interval of the abovementioned values) to reduce bitter taste when inhalation. In one embodiment, mixing the finely divided particle with the flavoring agent of the weight percentage of 1% has better efficiency for reducing bitter taste.

A pharmaceutical composition for dry powder inhalation is provided in some embodiments, including: an active ingredient and a first pharmacologically acceptable excipient. The active ingredient includes tadalafil or a pharmaceutically acceptable salt thereof. The first pharmacologically acceptable excipient includes amino acid, polysaccharide, phospholipid, polylactic acid, polylactic acid copolymer, or a combination thereof.

Through the use of the first pharmacologically acceptable excipient, the shape and the particle size of the pharmaceutical composition through spray dried can be regulated (including but not limited that the shape appears to be specific regular forms of spherical-shaped form or polyhedron form, and the particle sizes are more consistent in different lots), so that the aerosol property of the pharmaceutical composition can be improved.

In some embodiments, a viscosity of the first pharmacologically acceptable excipient is lower than 3 dl/g, such as from 0.1 dl/g to 3 dl/g. If the viscosity is too high, the first pharmacologically acceptable excipient is hardly spray dried and the particle size of the finely divided particle is too big to meet the requirement of inhalation administration.

In some embodiments, a weight percentage of the active ingredient is from 1% to 99% (such as 1%, 10%, 20%, 30%, 40%, 50%, 60%, 70%, 80%, 90%, 95%, 99% or any value between any interval of the abovementioned values), and a weight percentage of the first pharmacologically acceptable excipient is from 1% to 99% (such as 1%, 10%, 20%, 30% 40%, 50%, 60%, 70%, 80%, 90%, 95%, 99% or any value between any interval of the abovementioned values) based on 100% by weight of the active ingredient and the first pharmacologically acceptable excipient. If the weight percentage of the active ingredient is too low or the weight percentage of the first pharmacologically acceptable excipient is too high, the active ingredient provided in a specific unit of pharmaceutical composition is limited. If the weight percentage of the active ingredient is too high or the weight percentage of the first pharmacologically acceptable excipient is too low, the aerosol property of the pharmaceutical composition is reduced.

In some embodiments, a weight percentage of amino acid is from 1% to 99% (such as 1%, 5%, 10%, 15%, 20%, 30%, 40%, 50%, 60%, 70%, 80%, 90%, 99% or any value between any interval of the abovementioned values) based on 100% by weight of the active ingredient and the first pharmacologically acceptable excipient. If the weight percentage of amino acid is too high, the active ingredient provided in a specific unit of pharmaceutical composition is limited. If the weight percentage of amino acid is too low, the aerosol property of the pharmaceutical composition is reduced.

In some embodiments, a weight percentage of polysaccharide is from 1% to 99% (such as 1%, 5%, 10%, 15%, 20%, 30%, 40%, 50%, 60%, 70%, 80%, 90%, 95%, 99% or any value between any interval of the abovementioned values) based on 100% by weight of the active ingredient and the first pharmacologically acceptable excipient. If the weight percentage of polysaccharide is too high, the active ingredient provided in a specific unit of pharmaceutical composition is limited. If the weight percentage of polysaccharide is too low, the aerosol property of the pharmaceutical composition is reduced.

In some embodiments, a weight percentage of phospholipid is from 1% to 99% (such as 1%, 5%, 10%, 15%, 20%, 30%, 40%, 50%, 60%, 70%, 80%, 90%, 99% or any value between any interval of the abovementioned values) based on 100% by weight of the active ingredient and the first pharmacologically acceptable excipient. If the weight percentage of phospholipid is too high, the active ingredient provided in a specific unit of pharmaceutical composition is limited. If the weight percentage of phospholipid is too low, the aerosol property of the pharmaceutical composition is reduced.

In some embodiments, a weight percentage of polylactic acid (PLA) is from 1% to 50% (such as 1%, 5%, 10%, 15%, 20%, 30%, 40%, 50% or any value between any interval of the abovementioned values) based on 100% by weight of the active ingredient and the first pharmacologically acceptable excipient. If the weight percentage of PLA is too high, the active ingredient provided in a specific unit of pharmaceutical composition is limited. If the weight percentage of PLA is too low, the aerosol property of the pharmaceutical composition is reduced.

In some embodiments, a weight percentage of polylactic acid copolymer (such as PLGA) is from 1% to 50% (such as 1%, 5%, 10%, 15%, 20%, 30%, 40%, 50% or any value between any interval of the abovementioned values) based on 100% by weight of the active ingredient and the first pharmacologically acceptable excipient. If the weight percentage of polylactic acid copolymer is too high, the active ingredient provided in a specific unit of pharmaceutical composition is limited. If the weight percentage of polylactic acid copolymer is too low, the aerosol property of the pharmaceutical composition is reduced.

In some embodiments, the active ingredient and the first pharmacologically acceptable excipient forms a finely divided particle having a particle size of from 50 nm to 10 μm, such as 50 nm, 100 nm, 500 nm, 1 μm, 2 μm, 3 μm, 4 μm, 5 μm, 6 μm, 7 μm, 8 μm, 9 μm, 10 μm or any value between any interval of the abovementioned values. It is noted that the particle size of the finely divided particle is smaller than the active ingredient without spray drying. Therefore, the requirement of the particle size for inhalation to lungs will be fulfilled.

In some embodiments, the finely divided particle is provided in a solid spherical-shaped form, a hollow spherical-shaped form, a solid polyhedron form or a combination thereof. The shape of the finely divided particle can increase the flight distance of the finely divided particles and enhance the distribution ratio in lungs while administration.

In some embodiments, the pharmaceutical composition further includes a second pharmacologically acceptable excipient different from the first pharmacologically acceptable excipient, such as lactose, mannitol or a combination thereof. The addition of the second pharmacologically acceptable excipient can further increase the flight distance of the finely divided particles and enhance the distribution ratio in lungs while administration.

In some embodiments, a weight percentage of the second pharmacologically acceptable excipient is from 70% to 99.995% based on 100% by weight of the pharmaceutical composition, such as 70%, 80%, 90%, 95%, 99.995% or any value between any interval of the abovementioned values. If the weight percentage of the second pharmacologically acceptable excipient is too high, the active ingredient provided in a specific unit of pharmaceutical composition is limited. If the weight percentage of the second pharmacologically acceptable excipient is too low, the improvement of the aerosol property is limited. In some embodiments, when the pharmaceutical composition includes the second pharmacologically acceptable excipient, a total weight percentage of the active ingredient and the first pharmacologically acceptable excipient is from 0.005% to 30% (such as 0.005%, 0.01%, 0.1%, 1%, 10%, 20%, 30% or any value between any interval of the abovementioned values) based on 100% by weight of the pharmaceutical composition, and a weight ratio of the active ingredient and the first pharmacologically acceptable excipient is from 0.01:1 to 199:1, such as 0.01:1, 0.1:1, 1:1, 10:1, 20:1, 30:1, 40:1, 50:1, 60:1, 70:1, 80:1, 90:1, 100:1, 110:1, 120:1, 130:1, 140:1, 150:1, 160:1, 170:1, 180:1, 190:1, 199:1 or any value between any interval of the abovementioned values.

In some embodiments, the pharmaceutical composition further includes a third pharmacologically acceptable excipient for regulating the characteristics, such as aerodynamics, flavor, etc.

It should be understood that the above-described embodiments and the following examples are given by way of illustration, not limitation. Various changes and modifications within the scope of the present invention will become apparent to those skilled in the art from the present description.

For clarifying the pharmaceutical composition for dry powder inhalation and preparation method thereof, several examples and functional testes are described below in sequence.

### Example 1—Preparation Method of Pharmaceutical Composition for Dry Powder Inhalation and Physical Properties

1. First Pharmacologically Acceptable Excipients Added before Spray Drying

### (1) Amino Acid

Step a: Tadalafil was respectively dissolved in ethanol and leucine was dissolved in water, and then they were mixed together to form a mixture according to the formulation listed in Table 1.

TABLE 1

| Weight Ratio of Tadalafil and | Concentration of Tadalafil and |
|---|---|
| Leucine (w:w) | Leucine in the Mixture (w/v, %) |
| 5:95 | 2.0 |
| 50:50 | |
| 95:5 | |

Step b: The mixture obtained in step a was spray dried at an outlet temperature of 100° C. to form finely divided particles.

Compared with other conditions in Table 1, the finely divided particles prepared by the condition of the weight ratio of 50:50 and the concentration of 2.0% had the smaller particle size and more spherical-shaped form and were selected for the following physical observations.

### Shape of Finely Divided Particles

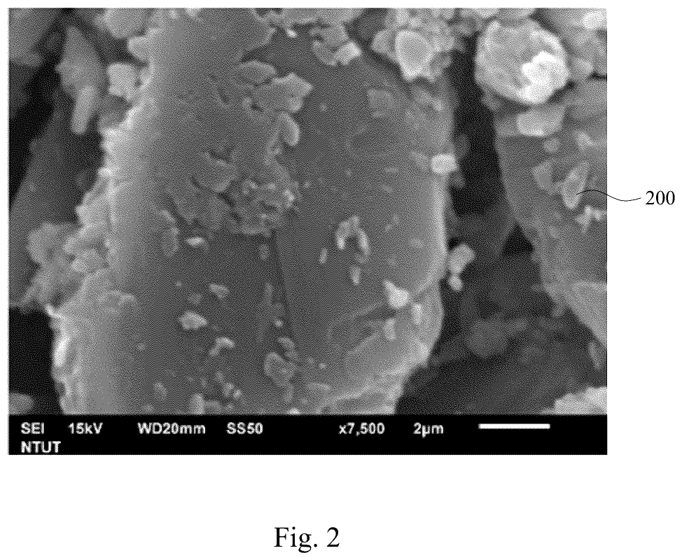

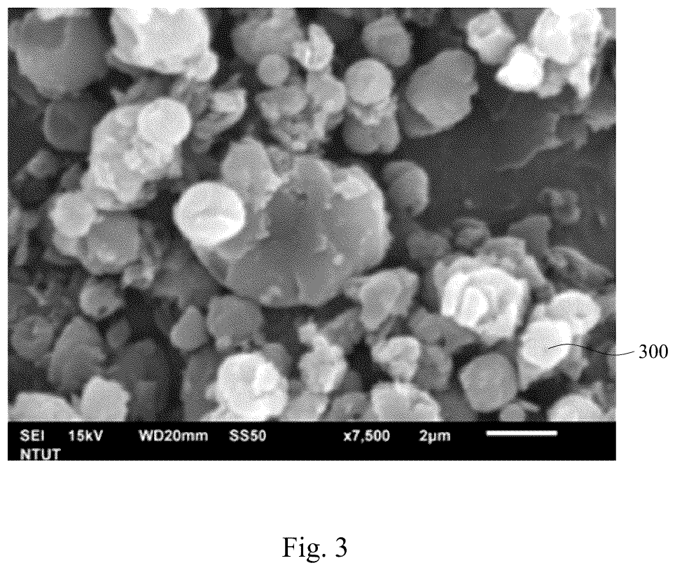

Appearances of tadalafil or finely divided particles in different preparation steps were observed under the field of an electron microscope, please refer to FIG. 2 and FIG. 3 for the results, in which tadalafil in FIG. 2 was observed at the step before step b (not spray dried), and finely divided particles in FIG. 3 was observed at the step after step b (spray dried), representing finely divided particles obtained by spray drying tadalafil and leucine.

FIG. 2 represented the shape of tadalafil 200 before step b (not spray dried) was irregular form, agglomeration appeared, and difference of the particle size was large. FIG. 3 represented the shape of finely divided particles 300 (spray dried) appeared to be spherical-shaped form or polyhedron form (such as golf ball type that was solid polyhedron form with multiple recesses in the surface). Compared with the irregular form of tadalafil 200 (FIG. 2) that was not spray dried, the shapes of finely divided particles 300 (FIG. 3) were more consistent.

### Particle Size

The particle size (D10, D50 and D90) of the finely divided particles was analyzed after 3 repeated tests and summarized in Table 2 (leucine was added).

TABLE 2
(leucine was added)

| | D10 (μm) | D50 (μm) | D90 (μm) |
|---|---|---|---|
| Mean | 2.41 | 3.46 | 6.30 |
| (3 repeated tests) | | | |

Note:
D10 referred to the particle size corresponding to the cumulative frequency of 10%.

D50 referred to the particle size corresponding to the cumulative frequency of 50%. D90 referred to the particle size corresponding to the cumulative frequency of 90%.

It was known that the particle size of more than 90% finely divided particles was generally smaller than 7 μm, D50 was only 3 μm to 4 μm, smaller than tadalafil that was not spray dried (tadalafil data was not shown in Table 2).

### (2) Polysaccharide

### (2)-1, Chitosan Glutamate

Step a: Tadalafil was dissolved in acetone, and chitosan glutamate (the molecular weight (Mw) of chitosan glutamate=5×10³ daltons to 190×10³ daltons) was dissolved in ethanol or acetone, Then, they were mixed together by using triple fluid nozzle according to the formulation listed in Table 3 and spray dried at an outlet temperature of 80° C. to form finely divided particles. Compared with the conventional dual fluid nozzle, a triple fluid nozzle could be used to better control the particle size and mixing uniformity when different solvents were used in the spray drying process.

TABLE 3

| | Concentration of Tadalafil and |
|---|---|
| Weight Ratio of Tadalafil and | Chitosan Glutamate in the Mixture |
| Chitosan Glutamate (w:w) | (w/v, %) |
| 20:80 | 2.0 |
| 50:50 | |
| 80:20 | |

Compared with other conditions in Table 3, the finely divided particles prepared by the condition of the weight ratio of 50:50 and the concentration of 2.0% had the smaller particle size and more spherical-shaped form. Therefore, the finely divided particles prepared by the condition of the weight ratio of 50:50 and the concentration of 2.0% were selected for the following physical observations.

### Shape of Finely Divided Particles

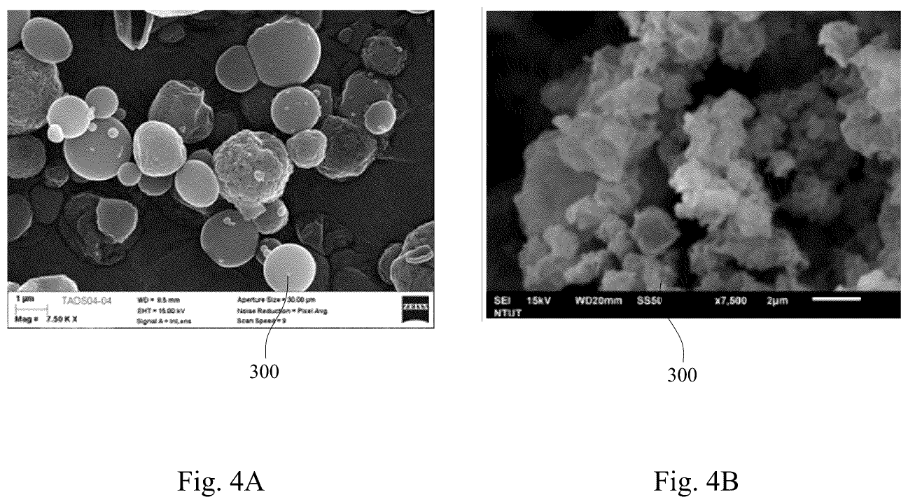

Appearances of finely divided particles (chitosan glutamate was served as the pharmacologically acceptable excipient) were observed under the field of an electron microscope, please refer to FIG. 4A for the results.

FIG. 4A represented the shape of finely divided particles 300 appeared to be spherical-shaped form (such as erythrocyte type). Compared with the irregular form of tadalafil 200 (FIG. 2) that was not spray dried, the shapes of finely divided particles 300 were more consistent.

### Particle Size

The particle size (D10, D50 and D90) of the finely divided particles was analyzed after 3 repeated tests and summarized in Table 4.

TABLE 4

| | D10 (μm) | D50 (μm) | D90 (μm) |
|---|---|---|---|
| Mean | 1.30 | 2.99 | 6.62 |
| (3 repeated tests) | | | |

It was known that the particle size of the finely divided particles was generally smaller than 7 μm, smaller than tadalafil that was not spray dried (not shown in Table 4).

### (2)-2, Hyaluronic Acid

Step a: Tadalafil was dissolved in ethanol and hyaluronic acid (molecular weight (Mw)-182.17 g/mole) was dissolved in water or 60% ethanol solution, and then they were mixed together to form a mixture according to the formulation listed in Table 5.

TABLE 5

| | Concentration of Tadalafil and |
|---|---|
| Weight Ratio of Tadalafil and | Hyaluronic acid in the Mixture |
| Hyaluronic acid (w:w) | (w/v, %) |
| 5:95 | 1.0 |
| 20:80 | |
| 95:5 | |

Step b: The mixture obtained in step a was spray dried at an outlet temperature of 100° C. to form finely divided particles.

Compared with other conditions in Table 5, the finely divided particles prepared by the condition of the weight ratio of 20:80 and the concentration of 1.0% had the smaller particle size and more spherical-shaped form. Therefore, the finely divided particles prepared by the condition of the weight ratio of 20:80 and the concentration of 1.0% were selected for the following physical observations.

### Shape of Finely Divided Particles

Appearances of finely divided particles (hyaluronic acid was used as pharmacologically acceptable excipient) were observed under the field of an electron microscope, please refer to FIG. 4B for the result.

FIG. 4B represented the shape of finely divided particles 300 appeared to be spherical-shaped form (such as erythrocyte-like type) or polyhedron form (such as golf ball type). Compared with the irregular form of tadalafil 200 (FIG. 2) that was not spray dried, the shapes of finely divided particles 300 were more consistent.

### Particle Size

The particle size (D10, D50 and D90) of the finely divided particles was analyzed after 3 repeated tests and summarized in Table 6.

TABLE 6

| | D10 (μm) | D50 (μm) | D90 (μm) |
|---|---|---|---|
| Mean | 1.30 | 2.99 | 6.62 |
| (3 repeated tests) | | | |

It was observed that the particle size of the finely divided particles was smaller than 7.0 μm, smaller than tadalafil that was not spray dried (not shown in Table 6).

### (3) Phospholipid

Tadalafil and DPSC (Mw=790 g/mol) were respectively dissolved in ethanol, and then they were mixed together at an outlet temperature of 90° C. to form a mixture according to the formulation listed in Table 7.

TABLE 7

| Weight Ratio of Tadalafil and DPSC | |
|---|---|
| (w:w) | DPSC in the Mixture (w/v, %) |
| 10:90 | 2.0 |
| 50:50 | |
| 90:10 | |

Compared with other conditions in Table 7, the finely divided particles prepared by the condition of the weight ratio of 50:50 and the concentration of 2% had the smaller particle size and more spherical-shaped form and were selected for the following physical observations.

### Shape of Finely Divided Particles

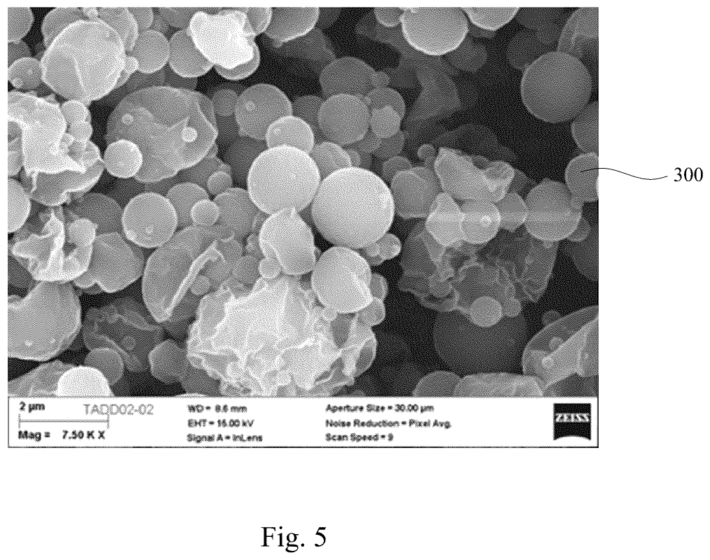

Appearances of the finely divided particles (DPSC was used as first pharmacologically acceptable excipient) after spray drying were observed under the field of an electron microscope, please refer to FIG. 5 for the results.

FIG. 5 represented the shapes of finely divided particles 300 appeared to be spherical-shaped form or polyhedron form (such as golf ball type). Compared with the irregular form of tadalafil 200 (FIG. 2) that was not spray dried, the shapes of finely divided particles 300 were more consistent.

### Particle Size

The particle size (D10, D50 and D90) of the finely divided particles that tadalafil was mixed with DSPC was summarized in Table 8.

TABLE 8
(DSPC was added)

| | D10 | D50 | D90 |
|---|---|---|---|
| | (μm) | (μm) | (μm) |
| Mean | 1.00 | 2.22 | 4.51 |
| (3 repeated tests) | | | |

It was observed that the particle size of the finely divided particles was generally smaller than 5 μm, smaller than tadalafil that was not spray dried (Tadalafil data was not shown in Table 8).

2. Second Pharmacologically Acceptable Excipients Added after Spray Drying

Lactose with two groups of particle sizes was added to a high shear mixer and mixed with the finely divided particles obtained by the abovementioned point 1. (3) that DPSC was used as the first pharmacologically acceptable excipient and the condition of the weight ratio of 50:50 and the concentration of 2% was selected, in which the mixing ratio of the second pharmacologically acceptable excipient (lactose) and the finely divided particles was conducted according to Table 9.

TABLE 9

| Material | Mixing Ratio |
|---|---|
| Lactose monohydrate A | 70% |
| (D50: 5 μm-50 μm, D90: 20 μm-80 μm) | |
| Lactose monohydrate B | 20% |
| (D50: 30 μm-125 μm, D90: 50 μm-300 μm) | |
| Finely divided particles | 10% |
| (DPSC was used as the first pharmacologically | |
| acceptable excipient) | |

### Aerosol Property

For compare the aerosol property of the finely divided particles prepared by directly spray drying, adding first pharmacologically acceptable excipient before spray drying, or adding first pharmacologically acceptable excipient before spray drying and second pharmacologically acceptable excipient after spray drying, the finely divided particles of group Tad only, group Tad+Leucine and group Tad+Leucine+Lactose were provided for detecting aerosol property according to cascade impaction (Cl) by using Next Generation Impactor (NGI) (brand: Copley Scientific, device name: Model 170) at a flow rate of 60 L/min. Group Tad only was prepared by directly spray drying tadalafil, group Tad+Leucine was prepared by adding first pharmacologically acceptable excipient, leucine, before spray drying (the better condition in Table 1 was selected, in which the weight ratio of tadalafil and leucine was 50:50, and the concentration of the mixture was 2.0%), and group Tad+Leucine+Lactose was prepared by adding first pharmacologically acceptable excipient, leucine, before spray drying and adding second pharmacologically acceptable excipient, lactose, after spray drying (the preparation ratio was similar to which of the abovementioned point 2).

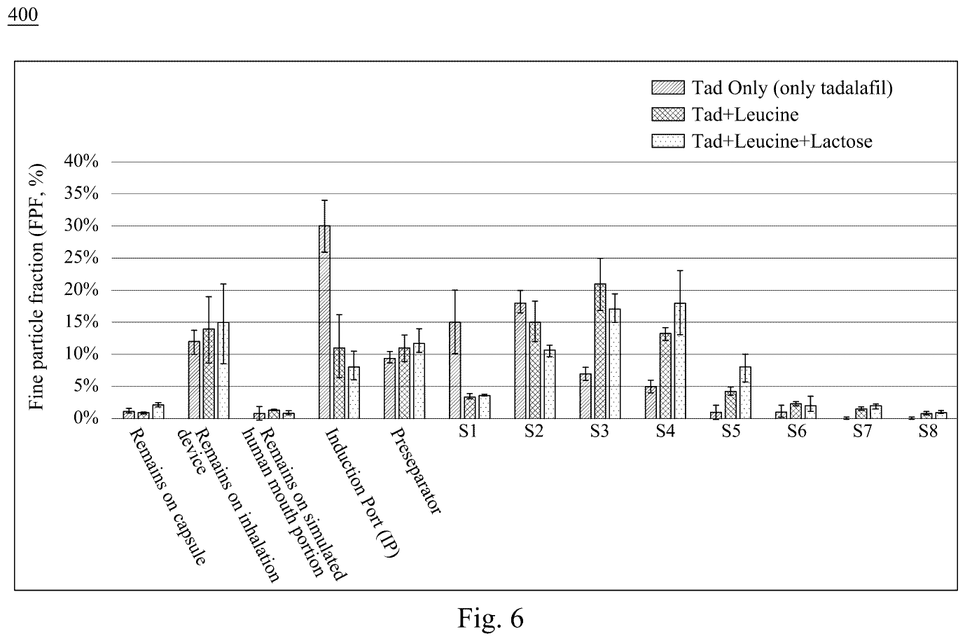

Please refer to FIG. 6 and Table 10 (the data summarized according to FIG. 6) for the results, in which fine particle fraction (FPF) was the percentage of the cumulative deposition content of the finely divided particles smaller than 5 μm to the total output content of the finely divided particles, which reflected the effective deposition ratio of the finely divided particles in the lungs.

FIG. 6 (comparison FIG. 400 of aerosol properties) represented that compared with group Tad only, the finely divided particles of group Tad+Leucine and group Tad+Leucine+Lactose were less distributed in the front stages (such as the stage of remains on capsule to the stage of S2) (the relatively shorter flight distance) and increasingly distributed in the following stages, such as the stages of S3 to S8, indicating the relatively longer flight distance was achieved. That is, compared with group Tad only, group Tad+Leucine and group Tad+Leucine+Lactose demonstrated the longer flight distance.

Furthermore, compared with group Tad+Leucine, it was observed that the finely divided particles of group Tad+Leucine+Lactose were increasingly distributed in the stages of S4 to S7, the stages that required the relatively longer flight distance. That is, compared with group Tad+Leucine, group Tad+Leucine+Lactose further demonstrated the longer flight distance.

TABLE 10

| Data Ave (n = 3) | Tad + Leucine + Lactose | Tad + Leucine | Tad only |
|---|---|---|---|
| Sample Recovery | 73.26 | 77.72 | 60.83 |
| (%) | | | |
| FPF(%) | 53.92% | 46.23% | 16.39% |
| MMAD (um) | 2.793 | 3.330 | 5.491 |

note:
MMAD was mass median aerodynamic diameter.

Table 10 represented group Tad+Leucine and group Tad+Leucine+Lactose performed higher FPF and smaller MMAD than group Tad only, and group Tad+Leucine+Lactose performed higher FPF and smaller MMAD than group Tad+Leucine.

Therefore, FIG. 6 and Table 10 indicated that the ranking of the flight distance (long to short) among the finely divided particles of the three groups was (1) the finely divided particles prepared by adding first pharmacologically acceptable excipient before spray drying and second pharmacologically acceptable excipient after spray drying, (2) the finely divided particles prepared by adding first pharmacologically acceptable excipient before spray drying, and (3) the finely divided particles prepared by directly spray drying tadalafil in order. That is, according to the data above, it was demonstrated that the finely divided particles prepared by adding first pharmacologically acceptable excipient or prepared by adding first pharmacologically acceptable excipient and second pharmacologically acceptable excipient achieved the higher distribution ratio in lungs than the finely divided particles prepared by directly spray drying.

Furthermore, please refer back to FIG. 3, FIG. 4A, FIG. 4B and FIG. 5 and refer to FIG. 6 and Table 10, together, it was also noted that the shapes of finely divided particles when tadalafil was at least mixed with first pharmacologically acceptable excipient before spray drying achieved the longer flight distance and the higher distribution ratio of the finely divided particles in lungs while administration.

Although the disclosure has been disclosed in the above embodiments, it is not intended to limit the disclosure, and it is to be understood that those skilled in the art can make various changes and modifications without departing from the spirit and scope of the disclosure. The scope of protection of the present disclosure is subject to the definition of the scope of claims.

What is claimed is:

1. A pharmaceutical composition for dry powder inhalation comprising:
an active ingredient, comprising tadalafil or a pharmaceutically acceptable salt thereof; and
a first pharmacologically acceptable excipient, comprising amino acid, polysaccharide, phospholipid, polylactic acid, polylactic acid copolymer, or a combination thereof.

2. The pharmaceutical composition of claim 1, wherein a weight percentage of the active ingredient is from 1% to 99% and a weight percentage of the first pharmacologically acceptable excipient is from 1% to 99% based on 100% by weight of the active ingredient and the first pharmacologically acceptable excipient.

3. The pharmaceutical composition of claim 1, wherein a weight percentage of the active ingredient is from 1% to 99%, and a weight percentage of the amino acid is from 1% to 99% based on 100% by weight of the active ingredient and the first pharmacologically acceptable excipient.

4. The pharmaceutical composition of claim 1, wherein the amino acid comprises glycine, alanine, valine, leucine, isoleucine, phenylalanine, tryptophan, tyrosine, aspartic acid, histidine, asparagine, glutamic acid, lysine, glutamine, methionine, arginine, serine, threonine, cysteine, proline or a combination thereof.

5. The pharmaceutical composition of claim 1, wherein a weight percentage of the active ingredient is from 1% to 99%, and a weight percentage of the polysaccharide is from 1% to 99% based on 100% by weight of the active ingredient and the first pharmacologically acceptable excipient.

6. The pharmaceutical composition of claim 1, wherein the polysaccharide comprises chitosan, chitosan salt, chitosan glutamate, hyaluronic acid or a combination thereof.

7. The pharmaceutical composition of claim 1, wherein a weight percentage of the active ingredient is from 1% to 99%, and a weight percentage of the phospholipid is from 1% to 99% based on 100% by weight of the active ingredient and the first pharmacologically acceptable excipient.

8. The pharmaceutical composition of claim 1, wherein the phospholipid comprises dipalmitoyl phosphatidylcholine, distearoyl phosphatidyl choline or a combination thereof.

9. The pharmaceutical composition of claim 1, wherein a weight percentage of the active ingredient is from 1% to 99%, and a weight percentage of the polylactic acid is from 1% to 50% based on 100% by weight of the active ingredient and the first pharmacologically acceptable excipient.

10. The pharmaceutical composition of claim 1, wherein a weight percentage of the active ingredient is from 1% to 99%, and a weight percentage of the polylactic acid copolymer is from 1% to 50% based on 100% by weight of the active ingredient and the first pharmacologically acceptable excipient.

11. The pharmaceutical composition of claim 1, wherein the polylactic acid copolymer comprises poly(lactic-co-glycolic acid).

12. The pharmaceutical composition of claim 1, wherein the active ingredient and the first pharmacologically acceptable excipient forms a finely divided particle having a particle size of from 50 nm to 10 μm.

13. The pharmaceutical composition of claim 12, wherein the finely divided particle is provided in a solid spherical-shaped form, a hollow spherical-shaped form, a solid polyhedron form or a combination thereof.

14. The pharmaceutical composition of claim 1, further comprising a second pharmacologically acceptable excipient different from the first pharmacologically acceptable excipient.

15. The pharmaceutical composition of claim 14, wherein a total weight percentage of the active ingredient and the first pharmacologically acceptable excipient is from 0.005% to 30%, and a weight percentage of the second pharmacologically acceptable excipient is from 70% to 99.995% based on 100% by weight of the pharmaceutical composition.

16. The pharmaceutical composition of claim 14, wherein the second pharmacologically acceptable excipient comprises lactose, mannitol or a combination thereof.

17. A method of preparing a pharmaceutical composition for dry powder inhalation, comprising:
dissolving an active ingredient in a first solvent to form a first solution, wherein the active ingredient comprises tadalafil or a pharmaceutically acceptable salt thereof;
dissolving a first pharmacologically acceptable excipient in a second solvent to form a second solution, wherein the first pharmacologically acceptable excipient comprises amino acid, polysaccharide, phospholipid, polylactic acid, polylactic acid copolymer, or a combination thereof;
mixing the first solution and the second solution to form a mixture; and
spray drying the mixture to form a finely divided particle.

18. The method of claim 17, wherein the first solvent comprises a first organic solvent, and the second solvent comprises a second organic solvent, water or a combination thereof.

19. The method of claim 17, wherein a weight percentage of the active ingredient and the first pharmacologically acceptable excipient is from 0.5% to 3% based on 100% by weight of the mixture.

20. The method of claim 17, wherein a weight ratio of the active ingredient and the first pharmacologically acceptable excipient in the mixture is from 0.01:1 to 199:1.

21. The method of claim 17, wherein spray drying the mixture is performed at an outlet temperature of from 35° C. to 110° C.

22. The method of claim 17, further comprising mixing the finely divided particle with a second pharmacologically acceptable excipient different from the first pharmacologically acceptable excipient.

23. The method of claim 22, wherein the second pharmacologically acceptable excipient comprises a first size group, a second size group or a combination thereof, wherein a volume-basis particle size distribution of the first size group is different from a volume-basis particle size distribution of the second size group.

24. The method of claim 23, wherein a particle size D50 of the first size group is from 5 μm to 50 μm and a particle size D50 of the second size group is from 30 μm to 125 μm.

25. The method of claim 17, further comprising mixing the finely divided particle with a flavoring agent.
